(19) United States
(12) Patent Application Publication
(10) Pub. No.: US 2021/0264346 A1
(43) Pub. Date: Aug. 26, 2021

(54) System for Improving Mine Safety and a Method for Using Same

(71) Applicant: Arizona board of regents on behalf of the university of Arizona, Tucson, AZ (US)

(72) Inventors: Moe Momayez, Tucson, AZ (US); Mary M. Poulton, Mead, WA (US)

(21) Appl. No.: 16/759,297

(22) PCT Filed: Oct. 25, 2018
(86) PCT No.: PCT/US18/57579

§ 371 Date: Apr. 24, 2020

Related U.S. Application Data
(60) Provisional application No. 62/577,084, filed on Oct. 25, 2017.

(51) Int. Cl. G06Q 10/06; A61B 5/02; G06N 3/08; G06K 9/62; G06F 1/16; G06Q 50/02

(52) U.S. Cl. CPC G06Q 10/063116; G06Q 10/063114; A61B 5/02; G06Q 50/02; G06K 9/6257; G06F 1/163; G06N 3/08

(57) ABSTRACT

A worksite safety system and method is disclosed. The system uses wearable sensors to collect biometric data from workers. The data are used to compute a worker's core body temperature on the basis of an individual profile associating historical biometric data with measured core body temperature. If the computed core body temperature crosses certain thresholds, alert actions are performed.

### CROSS-REFERENCE TO RELATED APPLICATIONS

This application claims priority to U.S. Provisional Patent Application No. 62/577,084, filed on Oct. 25, 2017, and entitled “A System for Worker Monitoring and Safety.”

### STATEMENT REGARDING FEDERALLY SPONSORED RESEARCH OR DEVELOPMENT

This invention was made with government support under Grant No. 200-2014-59953 awarded by the Center for Disease Control and Prevention. The government has certain rights in the invention.

### BACKGROUND

A variety of technologies are in the process of converging to enable significant advances in wearable biometric sensors. Continued advances in microelectronic sensors themselves, particularly microelectromechanical systems (MEMS), have combined with advances in packaging and integration (e.g., system on a chip (SOC) and system on glass (SOG), power storage, and networked communication (e.g., the Internet of Things (IOT)), to result in powerful wearable sensors and the ability to usefully analyze the data such sensors collect. While such technologies have already been employed in the consumer space (e.g., exercise and sleep monitoring sensors worn on the wrist), their application lags in industry and other fields.

Heat stress is one of the most significant physical hazards presented in any endeavor that requires sustained physical exertion. Heat stress adversely affects emergency response personnel (i.e., firefighters, police officers, paramedics, etc.), construction workers, agricultural workers, landscapers, road crews, mail carriers, delivery workers who work in open or un-air conditioned vehicles, factory floor workers, military personnel, athletes, oil and gas workers, and miners, just by way of example. Literally, anyone who works anywhere other than an air-conditioned office is potentially subject to heat stress.

One of the most demanding environments from a heat stress perspective is an underground mining operation. The underground mining environment is already hazardous due to risks such as unplanned explosions, equipment failure, work in proximity to heavy machinery, darkness, toxic gases and liquid solvents, and the presence of deep bodies of water. Mine work itself is physically demanding and puts inherent stress on the human body. As mines are worked at greater depths, temperatures increase due to geothermal heat factors, hotter ambient temperatures, incursion of high temperature ground water, and an overall lack of air circulation. Miners working in such high ambient temperature and relative humidity with radiant and conductive loading have a high risk of heat illnesses. Heat-related illnesses comprise a spectrum of progressive physiologic manifestations that result from excessive core body heat loading, including: heat cramps, heat rash, heat syncope, and heat exhaustion, and eventually, heat stroke. Further, heat stroke is a life-threatening medical emergency, resulting from excessively elevated core body temperature of >40° C., neurologic changes, and anhidrosis. In addition to resulting in emergent and acute conditions, heat strain incidents may increase workers' subsequent risk of and sensitivity to heat-related illnesses.

Aggravating these environmental factors is the fact that mining is a physically demanding activity that naturally results in higher body temperature. Worse yet, metal miners are required to work at higher metabolic rates than other miners. Studies comparing the crude incidence rate for heat illness per 1 million-person hours have found that the rate-ratio for heat illnesses in U.S. metal mines, between 1983-2001 was 61.1—significantly higher (p<0.001) than any other mine type. Evidence suggests that miners performing certain job tasks, of higher body mass index, and working day versus swing or night shift may be at even greater risk of heat illness. A recent pilot study by Applicants showed that an underground miner's heat illness risk is related to job-task, shift, and workers body mass index (BMI). Specifically, Applicants have identified that when miners performing taxing jobs were not properly hydrating, those working on swing shift were at increased risk for heat-related illnesses compared to day and night shift workers, and that the complex relationship between BMI and heat strain remains unclear.

Heat related incidents impose high costs on mining operations. According to different government and independent sources, the total direct and indirect cost of a single debilitating work site accident in the mining and construction industries can have a total economic impact of up to $5.4M. This includes financial costs, human costs (medical aid costs, rehabilitation costs, death benefits, bodily injury indemnities, income replacement indemnities), and productivity costs (loss of time and production). Currently, there is no single integrated technology available that provides for proper miner health and safety monitoring to help curb this exorbitant loss of revenue and productivity. While there are current proposals to measure worker core body temperature directly with internal sensors, these methods are cumbersome and intrusive. Additionally, while there are other current proposals to infer core body temperature of workers on the basis of measured environmental parameters (e.g., ambient air temperature), these systems are not particularly accurate, and do not account for variables unique to individual workers (e.g., the worker's own fitness level or body mass index (BMI). What is needed is an accurate, real-time method of easily measuring and predicting a worker's core body temperature, and taking remedial measures, so that heat related morbidity can be avoided.

### SUMMARY

In one embodiment, the invention relates to a system for predicting and inferring the core body temperature of a worker. In these embodiments, the worker wears a biometric sensor array, which collects and stores and/or transmits data relating to a variety of physiological parameters, for example, galvanic skin response, blood pressure, respiration rate and blood oxygen saturation. These parameters are associated with core body temperature, for an individual, under actual working conditions, through a training process. In the training process, a worker swallows an internal core temperature sensor, which sends data wirelessly to a computer, which time stamps and stores the data in association with time stamped data regarding the worker's physiological parameters, which are measured at the same time. The data are then used to train a neural network computer software program, which creates a weighted node network for an individual worker associating biometric data inputs with a prediction or inference of core body temperature. This computed neural network is then used on a real time basis to infer core body temperature on the basis of the same biometric inputs during a work situation.

In certain embodiments, a system is provided that performs certain action on the basis of a measurement of core body temperature as described above. This system includes a wearable sensor in networked electronic communication with a computer (i.e., a server), which collects and stores biometric information measured by the sensor. The server then applies the measured data to a trained neural network resulting in an inference of a worker's core body temperature. The server then checks the core body temperature determination against pre-set or dynamically set threshold ranges, and if the core body temperature measures fall within the ranges, takes certain actions. Exemplary ranges or thresholds might include the range between 38-39 deg. C., and 39-40 deg. C. When a core body temperature determination falls within a lower range, or crosses a lower threshold, the server takes a first action, for example, sending an alert message to a supervisor or the worker directly and/or directing the worker to take some remedial action such as to take a break. When a core body temperature determination falls within an upper range, or crosses an upper threshold, a second action is taken, for example, sending a second alert to a supervisor or the worker directly, and/or directing a worker or the worker's supervisor to evacuate the worker for medical attention. Other actions are also possible, such as adjusting air flow in a work space.

Thresholds and ranges, in certain embodiments, are dynamically determined on the basis of historical core body temperature. In certain embodiments, the system computes predicted core body temperature trajectories on the basis of current and historical data, and applies these trajectories to dynamically determined or pre-set thresholds and ranges to determine if and when to take alert or other actions. In certain embodiments, environmental data (e.g., air temperature, air flow rate, mine location, assigned worker task) is also used to determine core body temperature and/or temperature trajectories and alert ranges and thresholds.

While certain embodiments specify certain actions to be taken in response to instantaneous core body temperature determinations and/or calculated thresholds, other data may be analyzed with core body temperature/trajectory data to evoke another set of responses. For example, biometric data not strongly associated with core body temperature may be combined with core body temperature determinations to generate a measure of “likely worker impairment”, which may trigger other responses. Exemplary secondary biometric data indicative of worker well-being or impairment include gait, EEG readings, voice patterns, body position, and body location.

### BRIEF DESCRIPTION OF DRAWINGS

The technology disclosed herein will be better understood from a reading of the following detailed description taken in conjunction with the drawings in which like reference designators are used to designate like elements, and in which:

FIG. 1 is a schematic block diagram of a wearable sensor according to an embodiment of the invention;

FIG. 2A is a conceptual diagram of a neural net based individual worker profile according to an embodiment of the invention;

FIG. 2B illustrates a method of training an individual worker profile according to an embodiment of the invention;

FIG. 3 is a schematic block diagram illustrating an exemplary embodiment of a system for improving work site safety; and

FIG. 4 illustrates a method of ensuring worker safety according to an embodiment of the invention.

### DETAILED DESCRIPTION

Embodiments of the invention are directed to a system of widely distributed and networked sensors, which monitor both environmental and biological parameters for individual workers in a mining environment. Certain sensors are worn on the person of a miner. Others are in fixed and known locations within and around a mine. Still others are on mine equipment. The sensors are nodes in a mine-wide sensor network and may communicate wirelessly, to both fixed network gateways (in a hub and spoke network configuration), or in a peer-to-peer mesh network configuration with and through other sensors. Data from the sensor network is collected and compared to historical data to detect and predict hazardous conditions, for example, heat stress in a miner. Predictive heuristics are built by training a neural network expert system with sensor data and measured miner body temperature, allowing the system to predict a heat strain event based on collected environmental and miner specific data. The sensor network and neural network (also referred to herein as an “expert system”) are used to enhance mine safety by, for example, providing a lifeline network for evacuation and emergency response, ground stability detection, up-to-the-second geolocation tracking of personnel and equipment, and communication of a higher grade than the current industry standard, as well as detection of heat stress and other hazardous conditions for individuals.

The technology disclosed herein is described in one or more exemplary embodiments in the following description with reference to the Figures, in which like numbers represent the same or similar elements. Reference throughout this specification to “one embodiment,” “an embodiment,” or similar language means that a feature, structure, or characteristic described in connection with the embodiment is included in at least one embodiment of the present technology disclosed herein. Thus, appearances of the phrases “in one embodiment,” “in an embodiment,” and similar language throughout this specification may, but do not necessarily, all refer to the same embodiment.

The described features, structures, or characteristics of the technology disclosed herein may be combined in any suitable manner in one or more embodiments. In the following description, numerous specific details are recited to provide a thorough understanding of embodiments of the technology disclosed herein. One skilled in the relevant art will recognize, however, that the technology disclosed herein may be practiced without one or more of the specific details, or with other methods, components, materials, and so forth. In other instances, well-known structures, materials, or operations are not shown or described in detail to avoid obscuring aspects of the technology disclosed herein.

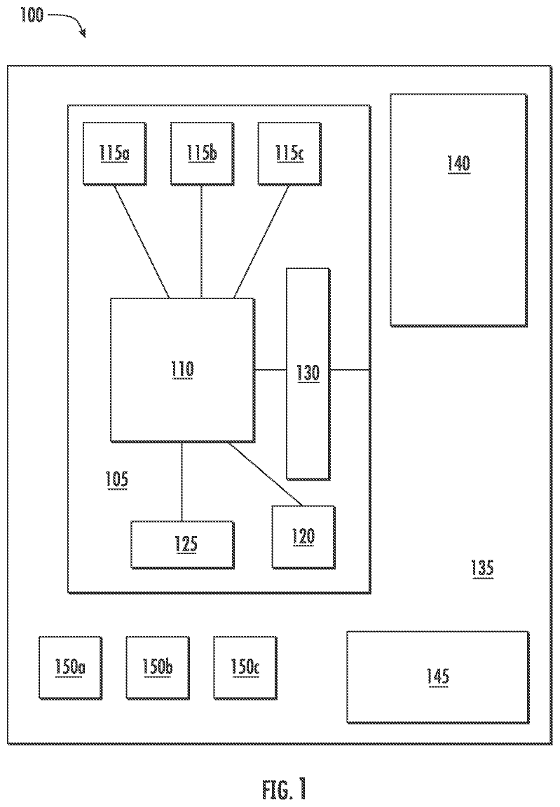

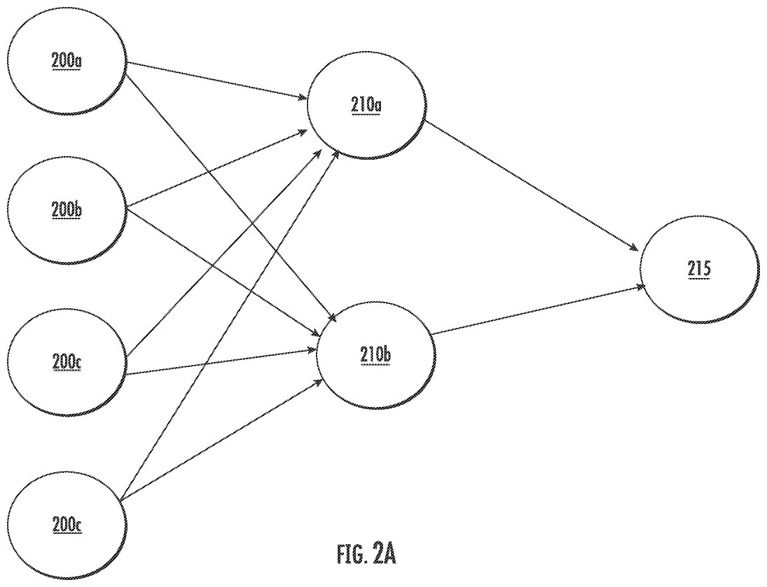

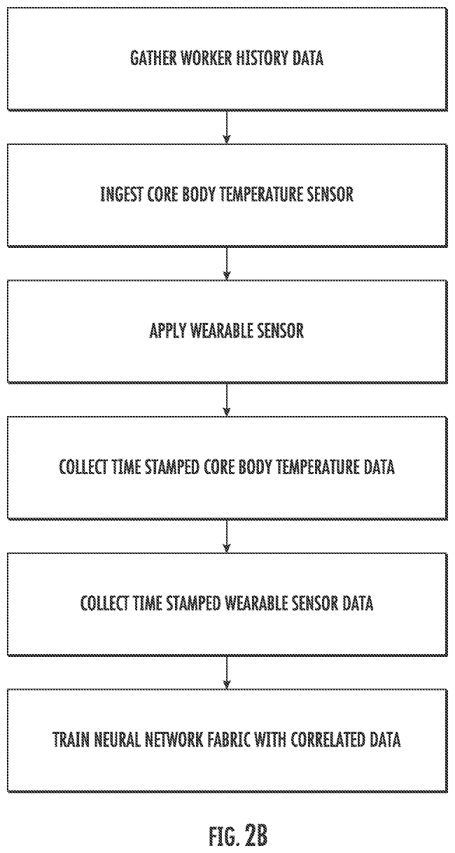

Referring to FIG. 1, there is shown a schematic block diagram of a wearable smart sensor 100 according to an embodiment of the invention. The sensor 100 of FIG. 2 includes an integrated sensor module 105, which includes a variety of application specific integrated circuits (ASICs) integrated on a common substrate such as a printed circuit board (PCB). Module 105 includes microprocessor 110 in electronic communication with a variety of sensors 115a-c. In one embodiment, sensors 115a-c include (1) a 3D accelerometer and 3D gyroscope, (2) a 3D magnetometer and 3D accelerometer and (3) a barometer. Microprocessor 110 is also in electronic communication with a MEMS-based microphone 120. Microprocessor 110 is also in communication with a wireless transceiver 125, which in one embodiment is a Bluetooth transceiver, which is itself in electronic communication with a non-illustrated antenna, for example, a Balun antenna. Microprocessor is also electronically connected to an I/O fabric or bus, which supports a variety of wired communications protocols such as USB, I2S/SPI, I squared C, as well as GPIO pins. An acceptable module 105 is the nMode wireless sensor module available from Samtec of 520 Park East Boulevard, New Albany, Ind., 47151.

Module 105 is integrated in a ruggedized, moisture resistant package with additional components on sensor board 135. Sensor board 135 components include additional external sensors 150a-c, DC power supply including a battery 145 and non-volatile memory (i.e., re-writable storage) 140 (e.g., eeprom or “flash” memory, hd card or similar). Sensor board 135 includes an additional, non-illustrated wired I/O interface such as a USB connector. All sensor board 135 components are in electronic communication with all module 105 components via the module's communication fabric 130. External sensors 150a-c may include, by way of example, anemometers, liquid or gas chemical sensors, accelerometers, magnetometers, optical instruments including light sources like LEDs to measure reflectance and transmittance, microphones, thermometers, and/or GPS or other geolocation receivers.

Sensor 100 may be configured to detect a variety of chemical, bio-physical conditions. Detectable conditions vary according to the sensors (115a-c; 150a-c) selected. Exemplary detectable conditions include: temperature, atmospheric pressure, acceleration, moisture and/or humidity, galvanic skin response, air flow velocity and/or volume, presence of certain gases, and vibration. When sensor 100 is worn next to a worker's skin, in one embodiment, it is configured to detect blood oxygen saturation, blood pressure, respiration rate (through accelerometry), and galvanic skin response.

One goal of a particular embodiment of a system using the sensor of FIG. 1 is to instantaneously compute a wearer's core body temperature. To accomplish, sense inputs are correlated with actual measurements of core body temperature according to known neural network training techniques, which are illustrated schematically in FIGS. 2A and B.

Neural networks are a known method of modeling complex systems, and therefore of predicting the outcomes of such systems on the basis of a large number of variables. Neural network analysis assumes that every input variable may contribute, solely or in complex interaction with other variables, in a particular outcome. Complex systems are, therefore, modeled as a weighted switch fabric, such as the fabric depicted in the schematic neural network of FIG. 2A. As is shown in FIG. 2A, a series of data inputs 200a-d is provided, which contribute, in an initially unknown manner to an output 215. A switch fabric is interposed between the input vector 200a-d and output 215. The switch fabric includes a number of nodes 210a-c, each of which received weighted contributions from each input 200a-d, and provide weighted contributions to output 215, the weighted contributions being represented in the figure by arrows. According to known methods, the weighting of the various nodes and connections can be computed for a given set of inputs and an output, and as more input and output data are received, the weights are adjusted to build an accurate model of the input-output relationship.

With regard specifically to the instant invention, in certain embodiments, measured biometric and other information, along with measured core body temperature information, is processed with artificial neural networks, appropriate statistical techniques (e.g. principal components analysis, discriminant analyses, etc), and related machine learning techniques such as Bayesian Belief Networks among others. Artificial or computational neural networks are machine learning architectures and paradigms that seek to find and analyze patterns in large data sets with methods that are roughly analogous to the way networks of biological neurons process sensory data. Architectures for artificial neural networks (ANN) can be divided between those that perform data classification without a user-provided solution (unsupervised learning, Kohonon networks or self-organizing maps for example) and those that perform either classification or estimation functions with user-provided training solutions (supervised learning, for example variations of feed-forward, fully-connected networks). Learning paradigms are formulas used to adjust connection weights between processing elements in the networks and can be purely mathematical optimization approaches (e.g. conjugate gradient) or can have more biological fidelity (e.g. adaptive resonance theory). The choice of architecture (the way processing elements and processing layers connect to each other) and the learning paradigms (the way data flows through the network and connection weights are changed) are a function of the problem to be solved.

Data from environmental and biosensors used to predict heat stress conditions may use unsupervised learning to cluster data to determine similarities and dissimilarities in data between individuals that are and are not experiencing discomfort from hot work environments. Data from these sensors will also be correlated with core body temperature measures and other short-term sensors (e.g. core body temperature sensors) using a supervised predictive neural network that can predict oncoming heat stress levels. Data can also be classified using supervised classification networks to determine stage of discomfort related to hot work environments. Networks of ANN can be used to assimilate data from environmental sensors and job task analyses sensors (cameras, kinematic measurements, etc.) to learn the relationship between physiologic states (body temperature, sweat rate, blood pressure, heart rate, brain waves, body mass index, stamina, etc.), environmental states (temperature, humidity, air flow, pollutants), and work states (slow-paced work, fast-paced work, work under loads, etc.). Machine learning algorithms “know” the world that they have been exposed to through the range of data variables used for training. Data are monitored to determine if they are still in the range of the training data and if not, data are collected and networks are re-trained with expanded data sets. The ANN, therefore, can continue to learn and adjust to the work environment.

FIG. 2B schematically illustrates the training methodology used in the instant invention. According to the method of FIG. 2B, first, a worker is given a questionnaire that elicits certain data regarding the worker's overall health and medical history. The questionnaire includes questions calculated to screen out subjects who are contraindicated for swallowing an internal temperature monitoring device (e.g., if the subject is very small, or has digestive conditions). More significantly for the purposes of the invention, the questionnaire also obtains data relevant for predicting core temperature on the basis of easily measured parameters, specifically, the worker's age, gender, weight, and habitual degree of hydration (i.e., how much water a person drinks every day). These data are used to train the neural network associated with the worker.

The worker then swallows an internal core body sensor that can communicate the worker's core body temperature, in real time, with an external computer. One suitable internal sensor is the CorTemp ingestible Bluetooth sensor available from HQInc., of 210 9th Street Dr., West Palmetto, Fla. 34221. When the core body temperature sensor is in position, the worker then puts on a wearable sensor, i.e., the sensor described above in relation to FIG. 1, and then works under normal working conditions. Core body temperature data from the ingested sensor, and the worn sensor, are collected wirelessly and stored in a time-correlated manner. This provides the input-output data training set, which is then used to train a neural network to generate a worker-specific profile that is capable of determining that individual's core body temperature on the basis of measured biometric parameters.

When this methodology has been used to build individual profiles for multiple subjects, patterns in the profiles will emerge, i.e., particularly important variables will be isolated, consistent weight patterns in the connection fabric, etc. On the basis of comparison of the individual profiles determined for multiple individuals, a generalized profile can be built that can approximate the heat response of an individual who has not yet gone through the calibration/training set forth above.

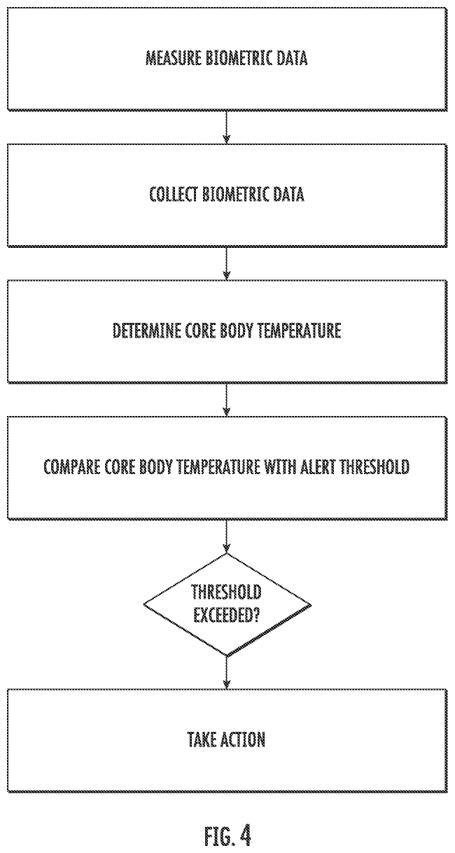

Once an individual worker's profile is calculated according to the method and system of FIGS. 2A and 2B, that profile and measured data can be used to determine a worker's core body temperature in real time and under real work conditions, and then direct some action on the basis of the core body temperature determination. FIG. 4 illustrates a method of ensuring worker safety according to an embodiment of the invention. According to the method of FIG. 4, a wearable sensor collects biometric information from a worker and transmits that information to a computer. Using the worker's individual profile, the computer computes the worker's core body temperature on the basis of the measured parameters. In one embodiment, the measured parameters are blood pressure, respiration rate, skin galvanic reaction and blood oxygen saturation, but other biometric and environmental parameters are possible, such as area air flow, air temperature, skin temperature, gait frequency, etc. The computed core body temperature is compared to one or more alert thresholds in order to determine the worker's condition. The thresholds may be preset and fixed, or they may be dynamically determined on the basis of a variety of measured personal, environmental and historical data. For example, if a worker has a history of heat stress events in the past, or is overweight, working a swing shift, or has been on shift for an extended amount of time, a lower temperature threshold may be applied than to a healthier worker.

Systems according to the invention direct some sort of remedial action in response to determination of an alert condition. Some types of remedial actions are discussed below, but these should not be considered limiting. Any remedial action is within the scope of the invention. Additionally, while some alert conditions are determined on the basis of core body temperature determination, others are determined on the basis of environmental data only, e.g., “get out” notifications in response to a determination that an environment is unsafe.

Referring still to FIG. 4, in one embodiment, when a worker's determined core body temperature exceeds a threshold (or alternatively or additionally, falls within an alert range), the system will have determined an alert condition and may perform some alert action in order to head-off a heat stress event. Exemplary alert actions will fall along a spectrum of severity, including information actions (a message being sent to the worker, or the worker's supervisor) regarding status, e.g., “you're getting hot.” A more severe action would include a directive message that directs the worker or supervisor to take some action. For example, the system may send an alert message (audibly, by email or SMS text to a worn device, audibly over an intercom system, etc.), to the worker directing the worker to take some remedial action. Exemplary actions include: taking a break, moving to a “cold space” in the work environment, leaving the work environment to seek medical attention, drinking water, using a cooling device such as a “cool vest”, immersing body parts in ice water, etc. Alert messages may also be sent to a worker's supervisor and/or logged electronically in an electronic data record associated with the worker (e.g., a personnel file). Additionally, automatic alert actions are also taken under certain circumstances. For example, when certain conditions are met, an audible alarm may be sounded, an ambulance called, or emergency personnel summoned, without any input from the worker. The severity of alert messages and remedial actions scales depending on the threshold crossed or the range into which a worker's core body temperature falls, and/or the presence of aggravating environmental conditions. For example, crossing a low temperature threshold (e.g., 37.5 degrees) might result in a “break” alert notice, while crossing a higher temperature threshold (e.g, 38.5 degrees) might result in a more serious “stop work and leave area” notice. Additionally, while environmental variables (e.g., air temperature, area heat flow, distance from help), are used, in some embodiments, to inform the determination of whether an alert condition exists, alternatively or additionally, environmental values are used only to determine the remedial action selected.

While the method above is described in terms of comparing an instantaneous (i.e., real-time) body temperature determination with thresholds or ranges, the invention is not so limited. Systems operating according to the invention can also compute likely core temperature trajectories based on current measured data, historical data, or a combination of the two. For example, systems according to the invention compute and store core body temperature data over time, resulting in a historical tend for an individual. These data can be used to extrapolate future trends by curve fitting to the already measured data, then extending the curve into the future to predict future core body temperature. Current trends can also be compared to historical trends for an individual to assist in predicting how a current trend is likely to progress. These computed trajectories may also be compared to fixed or dynamically determined thresholds or ranges, and alert events directed in advance of the point in time when heat stress is imminent. Additionally, while the methods described above rely on biometric data to compute core body temperature, other data may be used as well such as: environmental air temperature, air flow, time since last break, and task codes indicating the degree of physical challenge associated with a particular job task. In particular, environmental data may be used to determine the occurrence of an alert condition, independent of the individual biometric data and/or as a supplement to the biometric data. The opposite is also the case in certain embodiments: alert conditions are determined solely by data collected from worn body sensors, without reference to data collected from environmental sensors.

It is known that people suffer cognitive deficiency as a result of heat stress, even at core body temperature levels below 40-41 degrees, which is generally taken to constitute a heat emergency. Systems according to the invention rely on this observation to provide different types of alerts depending on a worker's core body temperature, with the goal being to remove the element of judgment from a worker as the worker's core body temperature moves closer to an emergent condition. According to this feature of the invention, when low core body temperature thresholds are crossed, a worker may be given a more permissive alert, e.g., a text message saying, “you are getting hot; maybe you should take a water break”, while as higher thresholds are crossed, the alerts are more in the nature of order, e.g., “stop work immediately; supervisor is being informed.”

While the inventive embodiments described above have been discussed in reference to individual workers, this should not be taken as limiting. Alert conditions can be determined with respect to groups of workers, for example, on the basis of environmental data like air temperature, and/or on the basis of core body temperature determinations from measured data from one or a sub-group of workers. Remedial alert actions are, in these cases, applied to groups of workers.

It will be appreciated by those of skill in the art that the basic principle of wearable worker sensors and trained, worker-specific neural net profiles, may have applications both beyond the issue of heat stress and beyond the mining environment. All such uses are within the scope of the invention. For example, systems according to the invention may be used to detect general worker impairment or unsafe working conditions. Using the geolocation features described more fully below, workers may be alerted when they have strayed into or near hazardous or out-of-bounds areas within the work environment. Relying on the observation above that cognitive function declines when core body temperature rises, heuristics that detect violations of no-go areas can change as a function of body temperature. For example, the system may maintain virtual “fence” boundaries around hazardous areas like roadways, tramways, areas where heavy equipment is active, blasting areas, pools of water or solvent, areas of bad air, etc. By tracking a worker's position, the worker can be warned away when approaching such areas, notations made in the worker's file, supervisors informed, etc. However, with real-time core temperature data, these fence boundaries can be pushed out, so that someone who is cognitively impaired by heat is warned away from a no-go area sooner and more firmly than someone not so impaired.

More generally, the sorts of biometric data useful for computing core body temperature may also be used to detect other useful pieces of information about the condition of a worker. For example, fatigue, cognitive impairment, injury or even physical shock may be determined by measuring and analyzing data regarding frequent or repeated violations of no-go areas, uneven or historically uncharacteristic gait, rapid breathing, blood pressure spikes or sweat in the absence of high core body temperature, changes in historical voice patterns (e.g., a worker is asked to repeat a calibrated test phrase, which is compared with historical recorded data), or changes in body position (e.g., worker is hunched over, inverted, or worn sensor is on the ground). Any or all of these data may be measured and analyzed to determine worker impairment, and alerts provided in response.

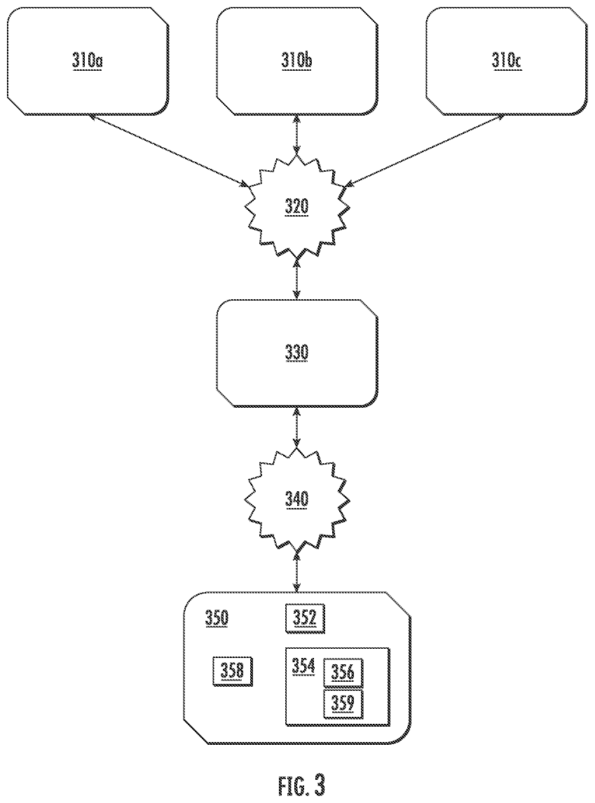

Referring now to FIG. 3, there is shown a schematic diagram for system 300 for improving worker safety according to an exemplary embodiment of the invention. The system of FIG. 3 is useful for collecting and analyzing data collected by the wearable sensors described in FIG. 1, above, and in issuing alerts in accordance with the method described in FIG. 4, but it has expanded functionality as well. In certain embodiments, the system comprises a plurality of nodes. As used herein, a node is defined as a sensor platform comprising a sensor and a power source, such as a battery. As is set forth above with respect to FIG. 1, a sensor can be embedded in a chip or otherwise integrated with a wired or wireless communication interface enabling connectivity to other nodes. In certain embodiments, the system includes a plurality of networked nodes such as environmental nodes, personal nodes (e.g., the wearable sensor 100 described above, including biophysical and biochemical sensors), and asset tracking nodes. Each environmental node 310a comprises an environmental sensor, each personal node 310b comprises a biophysical and/or biochemical sensor, and each asset tracking node 310c comprises an asset tracking sensor.

The system 300 comprises one or more gateways 330 and a server 350. As used herein, a gateway 330 is defined as a piece of networking hardware that has the following meaning: a gateway may contain devices such as protocol translators, impedance matching devices, rate converters, fault isolators, or signal translators as necessary to provide system interoperability. It also requires the establishment of mutually acceptable administrative procedures between both networks; and a protocol translation/mapping gateway interconnects networks with different network protocol technologies by performing the required protocol conversions. Moreover, the system 300 comprises the exemplary environmental node 310a that is communicatively connected to the gateway 330 through a communication fabric 320, the exemplary biophysical and biochemical node 310b that is also communicatively connected to the gateway 330 through communication fabric 320, and the exemplary asset tracking node 310c that is also communicatively connected to the gateway 330 through a communication fabric 320. In the system diagram of FIG. 3, communication fabric 320 is represented as a common communication fabric over which all exemplary nodes communicate with gateway 330, but this is not a requirement. Separate communication channels between nodes and gateway 330 are permissible.

Communication fabric 320 may be any physical communication medium carry signals according to any communications protocol capable of providing data communications between server 350 and nodes 310a-c. Exemplary communications media and standards include: wired (i.e., Ethernet, coaxial cable, optical fiber, powerline modulation) and wireless (WiFi, Bluetooth, UHF, LiFi, Leaker Feeder, etc.). In a preferred embodiment, communication fabric 320 comprises a wired Ethernet LAN including multiple wireless gateways in wireless communication with sensors 310a-c through, for example, Bluetooth or radio communication occurring in accordance with the 802.11 WiFi standards. In certain embodiments, communication fabric 320 is itself at least partially composed of additional nodes communicating in a peer-to-peer fashion through a mesh network. The gateway 330 is communicatively connected to the server 350 via a communication fabric 340, which has the same permissible characteristics as those described above with respect to communication fabric 320.

For the sake of clarity, FIG. 3 shows one environmental node 310a, one personal node 310b, one asset tracking node 310c, one gateway 330, and one server 350. FIG. 3 should not be taken as limiting. Rather, in other embodiments any number of entities and corresponding devices can be part of the system 300. In certain embodiments, the number of environmental nodes and the configuration of communications fabrics 320 and 340 are determined by the size and configuration of the working environment. In an exemplary environmental node distribution, one environmental node is disposed about 50 feet away from the next environmental node, but other distributions, densities and the number of environmental nodes are permissible depending upon work environment characteristics and the technology being used to support communications fabric 320.

In certain embodiments, the gateway 330 and the server 350 are each an article of manufacture. Examples of the article of manufacture include: a server, a mainframe computer, a mobile telephone, a smart telephone, a personal digital assistant, a personal computer, a laptop, a set-top box, an MP3 player, an email enabled device, a tablet computer, a web enabled device, or other special purpose computer each having one or more processors (e.g., a Central Processing Unit, a Graphical Processing Unit, or a microprocessor) that are configured to execute Applicants' API to receive information fields, transmit information fields, store information fields, or perform methods.

By way of illustration and not limitation, FIG. 3 illustrates the server 350 including a processor 352; a non-transitory computer readable medium 354 having a series of instructions 356 encoded therein; an input/output means 358, such as a keyboard, a mouse, a stylus, touch screen, a camera, a scanner, or a printer; and computer readable program code 359 encoded in non-transitory computer readable medium 354. Processor 352 utilizes computer readable program code 359 to operate server 350. In certain embodiments, server 350, in accordance with computer executable code 356, performs the neural network training, profile storage, data collection, analysis, decision, alert and storage functions described above with respect to FIGS. 1-2 and 4.

Environmental nodes 310a comprising environmental sensors are used to monitor environmental parameters of a work area such as but not limited to airflow, air pressure, temperature, relative humidity, ground stability, concentrations of particulate matter, and gases. Environmental nodes preferably include accelerometers, capable of measuring acceleration, which enables the detection of shifting walls, floors and ceilings, and may be useful to detecting or predicting slides or cave ins. Information/data regarding but not limited to identification/contribution of radiant and conductive sources, radon, diesel particulate matter (DPM), silica/coal dust (as appropriate), relative humidity, wind speed, wet bulb temperature, dry bulb temperature, dew point, barometric pressure, and water temperature are collected by the environmental sensors. In certain embodiments, each environmental node is communicatively connected via a communication fabric with each other.

In certain embodiments, environmental nodes 310a are distributed in fixed locations throughout a work environment, for example, on walls, floors and ceilings. Server, 350, in certain embodiments, has data stored thereon indicating the positions of environmental nodes 310a with respect to a fixed, predetermined coordinate system (e.g., latitude, longitude, altitude). In addition to accelerometry, monitoring the position of environmental nodes over time is useful in detecting and/or predicting shifting or instability in mine surfaces. In certain embodiments, environmental nodes of known positions are used to geolocate other nodes by known methods such as TDOA triangulation. This enables the time varying position of asset tracking and personal nodes to be determined, resulting in data about the position, velocity and acceleration of people and equipment within the mine. Such data may be used in conjunction with virtual fencing data to detect hazardous or inappropriate conditions, provide warnings (e.g., if an individual enters a hazardous or forbidden area, a truck driver exceeds a speed limit, or comes too close to another piece of equipment or wall, etc.), and reconstruct accidents.

Personal nodes (also referred to herein as bionodes) include biophysical and biochemical sensors (biosensors), and are worn by a mine worker. An exemplary bionode is described above with respect to FIG. 1. Biosensors collect information/data about a worker's physiological condition, such as vital signs (heart rate, respiration rate, blood oxygen saturation, perspiration rate, etc.) and skin temperature. Personal nodes also include sensors that collect data regarding an individual's immediate environment, such as immediate air temperature, oxygen concentration, presence of predetermined gases (e.g., diesel fumes), air pressure, etc. A data analysis system, such as server 350, or the ANA system described above with respect to FIG. 2, predicts core body temperature trends from data collected by environmental sensors and a person's personal sensor. This process may be made more accurate by calibrating skin temperature data to core body temperature by requiring a miner to wear a personal sensor after swallowing a calibration sensor that directly measures core body temperature, and correlating the resulting data. In addition, personal sensors may include multi-axis accelerometers, and may therefore generate time varying position data by known geolocation methods as discussed above, as well as expected trajectories and even data regarding the attitude (i.e., position) of an individual. This permits personal sensors to collect and/or generate data on the tempo with which an individual is working and gait, which data is used to predict trends in core body temperature, as well as data regarding impairment conditions and trajectories toward possible no-go zones.

In addition, asset tracking nodes comprising asset tracking sensors are used to collect information/data, such as geolocation tracking of mine equipment and data regarding the operation and condition of such equipment.

All collected information/data is transferred to gateway 330 via communication fabric 320. Gateway 330 transfers the collected information/data to server 350 via communication fabric 340. Alerts or other commands (programming, updates, voice prompts, messages, etc.) may be transferred from server 350 back down to the nodes via fabrics and gateways in the reverse process. To assist in this communication process, server 350 may further comprise one or more display screens. In certain embodiments, the nodes 310a-c, the gateway 330, and the sensor 350 include wired and/or wireless communication devices which employ various communication protocols including near field (e.g., “Bluetooth”) and/or far field communication capabilities (e.g., satellite communication or communication to cell sites of a cellular network) that support any number of services such as: telephony, Short Message Service (SMS) for text messaging, Multimedia Messaging Service (MMS) for transfer of photographs and videos, electronic mail (email) access, or Global Positioning System (GPS) service, for example. In certain embodiments, at least one of the communication fabrics 320 and 340 comprises the Internet, an intranet, an extranet, a storage area network (SAN), a wide area network (WAN), a local area network (LAN), a virtual private network, a satellite communications network, an interactive television network, or any combination of the foregoing. In certain embodiments, at least one of the communication fabrics contains either or both wired or wireless connections for the transmission of signals including electrical connections, magnetic connections, or a combination thereof. Examples of these types of connections include: radio frequency connections, optical connections, telephone links, a Digital Subscriber Line, or a cable link. Moreover, communication fabrics utilize any of a variety of communication protocols, such as Transmission Control Protocol/Internet Protocol (TCP/IP), for example.

With respect to data/information storage, in certain embodiments, said collected data/information is encoded in one or more hard disk drives, tape cartridge libraries, optical disks, combinations thereof, and/or any suitable data storage medium, storing one or more databases, or the components thereof, in a single location or in multiple locations, or as an array such as a Direct Access Storage Device (DASD), redundant array of independent disks (RAID), virtualization device, etc. In certain embodiments, said collected data/information is structured by a database model, such as a relational model, a hierarchical model, a network model, an entity-relationship model, an object-oriented model, or a combination thereof. In other embodiments, the said collected data/information is stored on the “Cloud” such as data storage library.

The results from the neural networks can be used to mitigate heat induced injuries. For example, the results can inform decision making using rule-based decision trees or other decision-making algorithms including graphical dashboards that alert human monitors to conditions that warrant attention or action. Decisions may be to temporarily move a worker to a cooler environment to recover, remove a worker for medical attention, change the work load (driving equipment versus manual operations of equipment), or change the environment (increase air flow, reduce temperature, reduce contaminants).

While the invention has been described primarily in connection with a mine environment, and particularly, an underground mine environment, the invention has broad applicability to many work environments where heat stress other worker impairments are an issue. Exemplary environments where embodiments of the invention may be advantageously employed include: open bit mines, oil and gas drilling sites, industrial facilities/factory floors, offshore oil rigs, agricultural enterprises, among emergency first responders like fire fighters, and athletics.

While the preferred embodiments of the present technology have been illustrated in detail, it should be apparent that modifications and adaptations to those embodiments may occur to one skilled in the art without departing from the scope of the present technology.

1. A method of improving worker safety, comprising:
providing a wearable biometric sensor worn by a worker;
collecting biometric data measured by the wearable biometric sensor during the course of work performed by the worker;
computing, on the basis of the collected biometric data, a physiological value for the worker;
comparing the computed physiological value with a computed or predetermined threshold;
solely on the basis of the comparison, determining and alert condition, and
taking an alert action.

2. The method of claim 1, wherein the physiological value is the worker's core body temperature.

3. The method of claim 2, wherein the biometric data measured includes one or more of: galvanic skin response, blood pressure, respiration rate and blood oxygen saturation.

4. The method of claim 2, wherein the biometric data measured does not include worker skin temperature.

5. The method of claim 2, further comprising computing an individual worker profile correlating historical measured biometric data with historical measured core body temperature, and wherein computing the physiological value for the worker includes using the individual worker profile to compute the physiological value on the basis of newly measured biometric data.

6. The method of claim 5, wherein computing an individual worker profile includes training a neural network.

7. The method of claim 2, wherein taking an alert action comprises directing the worker to do one or more of the following: take a break, change work tasks, use a cooling off device, and leave a work area.

8. The method of claim 2, wherein computing, on the basis of the collected biometric data, a physiological value for the worker, includes computing a projected trajectory for the physiological value over time.

9. The method of claim 2, wherein taking an alert action comprises one or more of: sending an information message, sending a command and taking an automatic action.

10. The method of claim 2, wherein taking an alert action comprises sending a message to individuals other than the worker.

11. The method of claim 1, further including measuring non-biometric environmental parameters, and computing the physiological value, in part, on the basis of the measured non-biometric environmental parameters.

12. The method of claim 1, wherein the alert action is determined on the basis of both measured biometric parameters and measured environmental parameters.

13. A system for improving worker safety, comprising:
a wearable biometric sensor, worn by a worker;
a network gateway in communication with the wearable biometric sensor;
a server in communication with the sensor via a communications fabric and the gateway, the server comprising a programmable processor and non-volatile storage including computer executable instructions capable of causing the programmable processor to:
receive biometric data from the wearable biometric sensor;
compute a physiological value for the worker;
compare the physiological value with a predetermined or computed threshold;
on the basis of the comparison, determining an alert condition, and
taking an alert action.

14. The system of claim 13, wherein the non-volatile storage includes computer executable instructions representing an individual worker profile correlating historical measured biometric data with historical measured physiological values, and wherein, the physiological value is computed on the basis of this profile.

15. The system of claim 14, wherein the physiological value is the worker's core body temperature, and wherein the received biometric data includes data relating to one or more of the worker's: blood pressure, respiration rate, blood oxygen saturation and galvanic skin response.

16. The system of claim 14, wherein the wearable biometric sensor includes a processor, one or more sensors, a power supply and a wireless communications interface.

17. The system of claim 16, wherein the one or more sensors include sensors for measuring one or more of: blood pressure, respiration rate, blood oxygen saturation and galvanic skin response.

18. A worksite safety system, comprising:
a plurality of nodes, each node including a sensor capable of measuring environmental data, a power source and a communications interface, the plurality of nodes including at least one environmental node comprising at least one environmental sensor and at least one personal node worn on a miner, the personal node having a biophysical and biochemical sensor that collects data regarding a worker's physical condition;
a gateway; and
a server, wherein the plurality of nodes transmits information collected by sensors to the server via the gateway;
wherein:
the system uses the information collected by sensors to predict trends in the core body temperature of a worker wearing the personal node.

19. The system of claim 18, wherein the worksite is an underground mine, and wherein the plurality of nodes includes a plurality of environmental nodes disposed on the walls, floor or ceiling of an underground mine, and wherein, the nodes wirelessly communicate with one another in a mesh network.

20. The system of claim 18, wherein the plurality of nodes further comprises at least one asset tracking node having an asset tracking sensor.

21. The system of claim 18, wherein the system, if a predicted core body temperature trend crosses a predetermined threshold, causes a mitigation action to be taken.

22. A non-transitory computer usable medium encoded with a computer program product to improve worksite safety and usable with programmable computer processor disposed within a computer, comprising:
computer readable program code which causes said programmable computer processor to perform the following steps:
collect biometric data collected by the wearable biometric sensor during the course of work performed by the worker;
compute, on the basis of the collected biometric data, the core body temperature of the worker;
compare the computed physiological value with a computed or predetermined threshold;
on the basis of the comparison, determining an alert condition, and
taking an alert action.
